(19) United States
(12) Patent Application Publication
(10) Pub. No.: US 2023/0338068 A1
(43) Pub. Date: Oct. 26, 2023

(54) HUMERAL REPAIR IMPLANT

(71) Applicant: Ritesh A Rathi, Maninagar (IN)

(72) Inventor: Ritesh A Rathi, Maninagar (IN)

(21) Appl. No.: 17/755,350

(22) PCT Filed: Jun. 24, 2021
(86) PCT No.: PCT/IB2021/055608

§ 371 Date: Apr. 27, 2022

(30) Foreign Application Priority Data
Jun. 24, 2020 (GB) 2009659.0

(51) Int. Cl. A61B 17/72

(52) U.S. Cl. CPC A61B 17/7283; A61B 17/7233; A61F 2002/2853

(57) ABSTRACT

The present disclosure envisages a humeral repair implant for repair of a fracture of the proximal humerus. The implant comprises a hollow body, a conical section, and an operative lower section. The hollow body is open at both ends. The conical section is configured at its top portion. The operative lower section is extending downwards from the conical section. A plurality of perforations is defined in the conical section. The perforations are configured to communicate between the inside and outside of the conical section.

### CROSS-REFERENCE TO RELATED APPLICATIONS

This application is a National Stage application of International Application No. PCT/IB2021/055608 filed on Jun. 24, 2021, which claims priority to Great Britain Application No. 2009659.0 filed Jun. 24, 2020.

### BACKGROUND

### Field

The present disclosure relates to the field of medical implants for the repair of fractures of the proximal humerus.

### Discussion of Related Art

As used in the present disclosure, the following terms are generally intended to have the meaning as set forth below, except to the extent that the context in which they are used indicate otherwise.

The term “Anatomical Neck” refers to a bone that is obliquely directed, forming an obtuse angle with the body of the humerus.

The term “Humerus Head” refers to a large rounded head that articulates with the glenoid cavity of the scapula forming the glenohumeral joint.

The term “Greater Tuberosity” refers to prominent area of bone at the top of the humerus and is the attachment for the two large, powerful rotator cuff muscles-supraspinatus and infraspinatus.

The term “Lesser Tuberosity” refers to the anterior of the two tubercles of the neck of the humerus on which the subscapularis is inserted.

The term “Surgical Neck” refers to the constricted part of the humerus just below the tuberosities (i.e., greater and lesser tuberosities).

FIG. 1 illustrates, for reference, the proximal end of a human humerus, generally indicated by 1. The anatomical features illustrated include the head 2 of the humerus, an irregular hemispherical surface that forms the articulating surface of the “ball” of the ball-and-socket shoulder joint. Also illustrated are the greater tuberosity 3, lateral to the head 2, and the lesser tuberosity 4, inferior to the head 2. The “anatomical neck” 5 of the humerus is the region between the tuberosities 3, 4 and the head 2. The “surgical neck” 6 is region between the tuberosities 3, 4 and the shaft 7 of the humerus. The region between the tuberosities 3, 4 is referred to as the intertubercular groove 8.

FIG. 2 illustrates a common fracture of the proximal humerus that might occur following a fall, especially by an individual having weakened bones, e.g. through osteoporosis. The humerus 1 breaks into essentially four sections, the greater 3 and lesser 4 tuberosities, the head 2 and the shaft 7. The fractures planes correspond to the anatomical 5 and surgical 6 necks, and the intertubercular groove 8.

A typical method of repairing such a fracture, is by the insertion of a replacement ball, mimicking the head of the humerus. This is mounted on a shaft which extends into the interior of shaft 7 of the humerus. Such a repair results in a metal-to-bone interface (i.e. new metal ball to existing socket) which is not ideal. Additionally, the tuberosities act as attachment points for a number of muscles that make up the rotator cuff of the shoulder. The use of an artificial joint to repair such a fracture can lead to problems with these muscles and their attachment points.

Therefore, there is felt a need of a humeral repair implant that alleviates the aforementioned drawbacks.

### SUMMARY

Some of the objects of the present disclosure, which at least one embodiment herein satisfies, are as follows:

An object of the present disclosure is to provide a humeral repair implant.

Another object of the present disclosure is to provide a humeral repair implant, and a method of repair of a proximal humeral fracture that preserves the original anatomical elements of the joint, rather than replacing them.

Other objects and advantages of the present disclosure will be more apparent from the following description, which is not intended to limit the scope of the present disclosure.

The present disclosure envisages a humeral repair implant for repair of a fracture of the proximal humerus. The implant comprises a hollow body, a conical section, and an operative lower end. The hollow body is open at both ends. The conical section is configured at its top portion. The operative lower section is extending downwards from the conical section. A plurality of perforations defined in the conical section. The perforations are configured to communicate between the inside and outside of the conical section.

In an embodiment, the operative lower section is tubular.

In an embodiment, the conical section and the operative lower section are separated by a step.

In an embodiment, the plane of the open end of the conical section is angled relative to the axis of the operative lower section. In another embodiment, the angle is between 15° and 45°.

In an embodiment, the cross-section of the conical section perpendicular to the axis of the conical section is circular.

In an embodiment, the cross-section of the conical section perpendicular to the axis of the operative lower section is in the form of an isosceles trapezium having rounded vertices.

In an embodiment, a slot is configured at the bottom open end extending towards the step between the conical section and the lower section.

In an embodiment, the outside surface of the operative lower section is provided with protruding ridges.

In an embodiment, at least a portion of the outwardly-facing surface of the operative lower section is roughened.

In an embodiment, at least a portion of the outwardly-facing surface of the operative lower section is a trabecular surface.

In an alternate embodiment, the conical section and the operative lower section are formed of a single curved surface extending from top to bottom portion of the implant.

In an alternate embodiment, the operative lower section is provided with perforations communicating between the inside and outside.

In an alternate embodiment, a plurality of serrations facing downwards is configured on the operative lower section.

In an embodiment, the implant is made from any suitable material including metal, biocompatible plastics material or biodegradable material.

### BRIEF DESCRIPTION OF THE DRAWINGS

A humeral repair implant of the present disclosure will now be described with the help of the accompanying drawing, in which:

FIG. 1 illustrates the proximal end of a humerus;

FIG. 2 illustrates a typical fracture in the proximal end of a humerus;

FIGS. 3-5 illustrate a front view of a humeral repair implant, in accordance with an embodiment of the present disclosure;

FIG. 6A illustrates a top view of the humeral repair implant, in accordance with an embodiment of the present disclosure;

FIG. 6B illustrates a front view of the humeral repair implant of FIG. 6A;

FIG. 6C illustrates a bottom view of the humeral repair implant of FIG. 6A;

FIG. 6D illustrates an isometric view of a lower section of the implant of FIGS. 6A-6C;

FIG. 7A illustrates a top view of the humeral repair implant, in accordance with an embodiment of the present disclosure;

FIG. 7B illustrates a front view of the humeral repair implant of FIG. 7A;

FIG. 7C illustrates a bottom view of the humeral repair implant of FIG. 7A;

FIG. 7D illustrates, in perspective view, the parallel-sided portion of the implant of FIGS. 7A-7C;

FIGS. 8A-8C illustrates stages in a procedure to insert an implant into the shaft of a humerus;

FIGS. 9-12 illustrate stages in a procedure to attach bone portions to an implant;

FIG. 13 illustrates attachment of the head of a humerus to a nail inserted into the shaft of a humerus;

FIG. 14 is a cross-section of FIG. 13 along the line A-A;

FIG. 15 illustrates a front view of a humeral repair implant in accordance with an alternate embodiment of the present disclosure;

FIG. 16 illustrates a side view of the humeral repair implant of FIG. 15;

FIG. 17 illustrates a top isometric view of the humeral repair of FIG. 15;

FIG. 18 illustrates a top view of the humeral repair of FIG. 15;

and

FIG. 19 illustrates a bottom isometric view of humeral repair of FIG. 15.

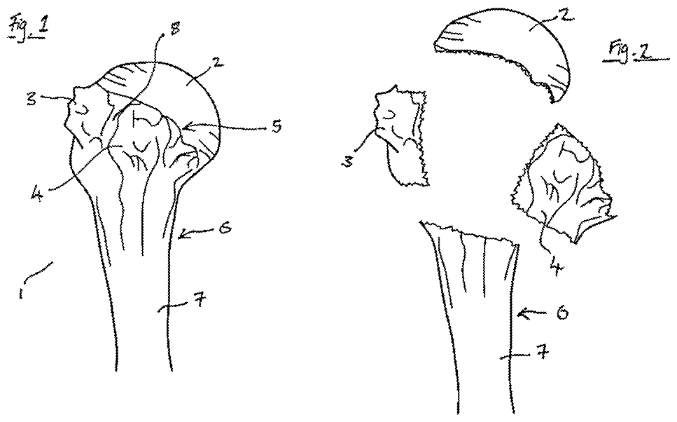

### DETAILED DESCRIPTION OF THE EMBODIMENTS

1—Humerus
2—Head
3—Greater tuberosity
4—Lesser tuberosity
5—Anatomical neck
6—Surgical neck
7—Shaft of humerus
8—Intertubercular groove
9—Humeral repair implant
H—Hollow body
10—Lower section
11—Conical section
12—Perforations
13—Plane of open end
14—Step
15—Ridges
15A—Serrations
16—Leading end
17—Slot
18—Proximal end
19—Internal surface
20—Rod
21—Threaded hole
22, 24—Screw
23—Plate

Embodiments, of the present disclosure, will now be described with reference to the accompanying drawing.

Embodiments are provided so as to thoroughly and fully convey the scope of the present disclosure to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the”, may be intended to include the plural forms as well, unless the context clearly suggests otherwise. The terms “including,” and “having,” are open ended transitional phrases and therefore specify the presence of stated features, operations, integers, elements and/or components, but do not forbid the presence or addition of one or more other features, operations, integers, elements, components, and/or groups thereof.

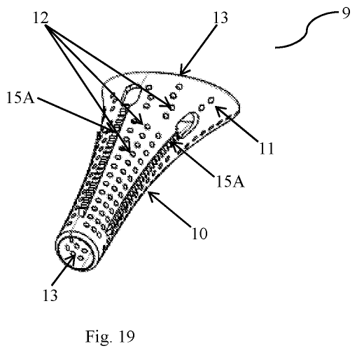

The present disclosure envisages a humeral repair implant 9 for repair of a fracture of the proximal humerus. The implant 9 (hereinafter referred as “implant 9”) is now described with reference to FIG. 3 through FIG. 19.

The implant 9 comprises a hollow body H, a conical section 11, and an operative lower end 10. The hollow body is open at both ends. The conical section 11 is configured at the top portion of the hollow body H. The operative lower section 10 is extending downwards from the conical section 11. The conical section 11 includes a plurality of perforations 12 defined therein. The perforations 12 are configured to communicate between the inside and outside of the conical section 11. These perforations 12 are used to receive sutures to enable bone portions to be attached to the implant 9.

In an embodiment, the operative lower section 10 is tubular. In an embodiment, the implant 9 is made from titanium, a titanium alloy, cobalt chrome, or another suitable biocompatible material including metal, biocompatible plastics material or biodegradable material. In an embodiment, the implant 9 made of biodegradable material, so that the material of the implant 9 degrades or decomposes after the reconstructed humerus has healed. In an embodiment, the conical section 11 and the operative lower section 10 are separated by a step 14 (please refer to FIGS. 3 to 7C).

In an embodiment, at least a portion of the outwardly-facing surface of the conical section 11 is a trabecular surface to aid longer-term securement of the bone to the implant 9. The trabecular surface will promote bone growth and attachment of the tuberosities to the tapered region. The trabecular surface treatments on the operative lower section 10 of the implant 9 are not required, or desired, to prevent bone growth on the inside face of the shaft of the humerus. In an embodiment, at least a portion of the outwardly-facing surface of the operative lower section 10 is roughened.

The plane of the open end 13 of the conical section 11 is angled relative to the axis of the operative lower section 10 (please refer to FIG. 3). The angle is chosen to be approximately the same as the angle of the anatomical neck 5 of the humerus 1 relative to the cross-sectional plane of the shaft 7 of the humerus, between 15° and 45°, such as between 25° and 35°, i.e. about 30°.

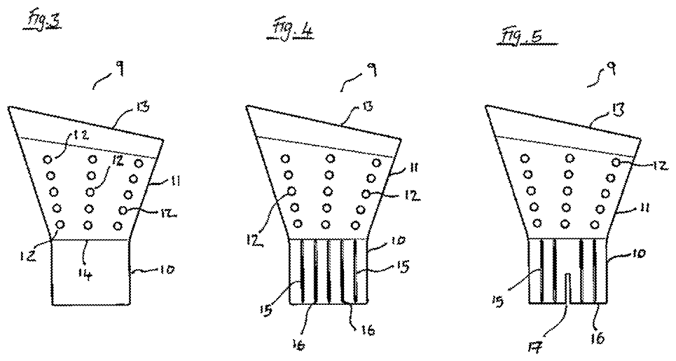

The outside surface of the operative lower section 10 is provided with protruding ridges 15 (please refer to FIG. 4). The protruding ridges 15 are running parallel to the axis of the lower section 10. When the implant 9 is in place in the shaft 7 of a humerus 1 (as will be described below), the ridges 15 engage with the interior surface of the bone to resist rotational movement of the implant 9 relative to the humeral shaft 7. In preferred embodiments, the leading end 16 of each ridge 15 terminates in a point, or sharp edge, to assist the ridges 15 in forming a channel on the inside surface of the humeral shaft 7 (bone) when it is inserted.

The implant 9 is further provided with a slot 17 (please refer to FIG. 5) at the open end 13 of the lower section 10 extending through the wall of the implant 9. The slot 17 is configured to be parallel to the axis of the conical section 11, and to extend part-way thereup. The slot 17 is configured to allow a small amount of reduction in the circumference of the lower section 10, when it is inserted, thereby reducing the chance of unnecessary trauma to the shaft 7 of the humerus 1.

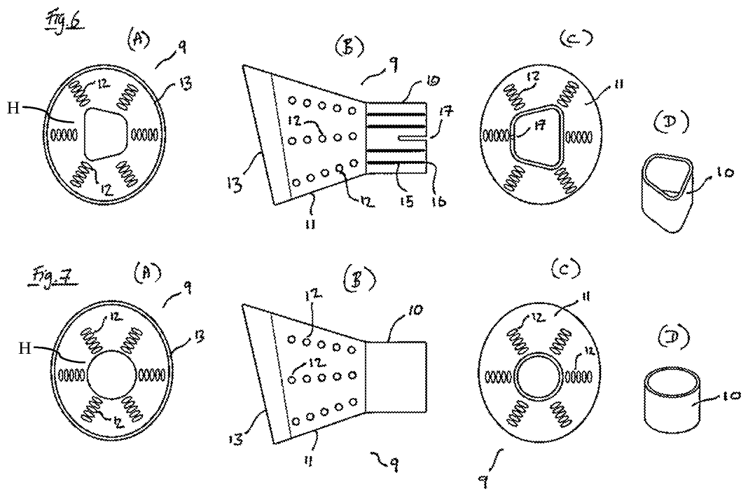

The cross-section of the conical section 11 is perpendicular to the axis of the operative lower section 10 and is in the form of an isosceles trapezium having rounded vertices (please refer FIGS. 6A-6C). FIG. 6D depicts a constant cross-section of the lower section 10 along its length.

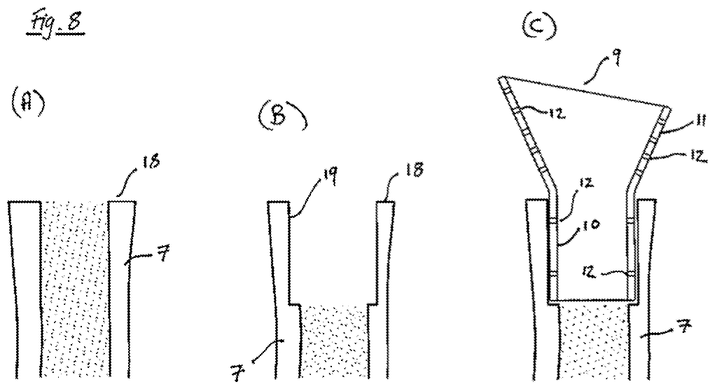

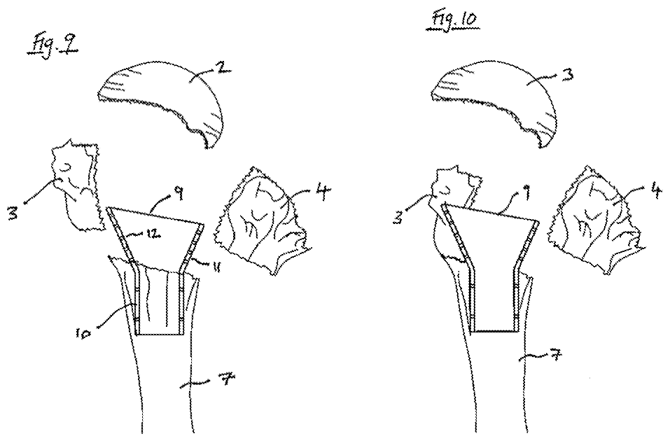

The cross-section of the conical section 11 perpendicular to the axis of the conical section 11 is circular (please refer FIGS. 7A-7D). In embodiments of the invention, the cross-sectional shape of the lower section 10 is formed to more closely match the interior cavity of a typical humerus shaft 7, e.g. having a profile somewhere between the illustrated isosceles trapezium and a circle.

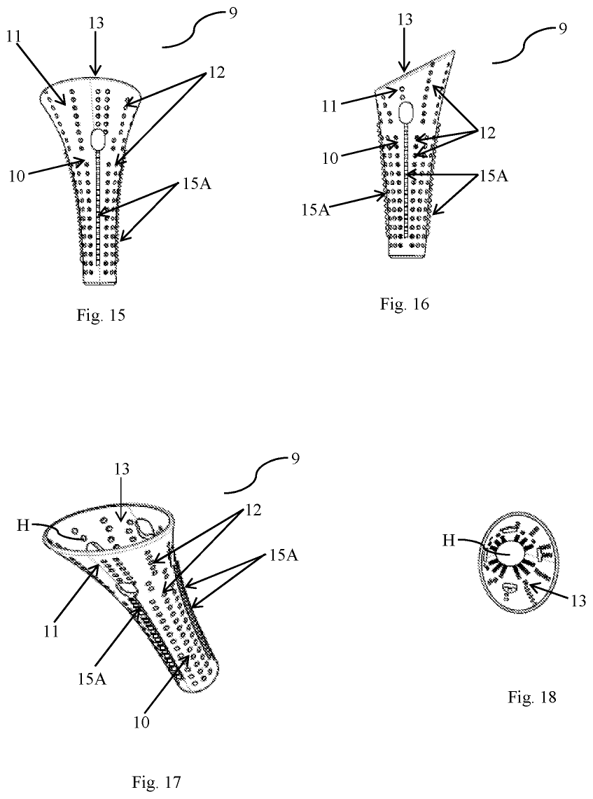

The present disclosure also envisages an alternate embodiment of the implant 9. FIGS. 15-19 envisages an alternate embodiment of the implant 9 which is analogous to the implant 9 of FIGS. 3-7C in functionality. The implant 9 of FIGS. 15-19 also comprises a hollow body H, a conical section 11, and an operative lower section 10. The hollow body is open at both ends. The conical section 11 is configured at the top portion of the hollow body H. The operative lower section 10 is extending downwards from the conical section 11. The conical section 11 includes a plurality of perforations 12 defined therein. The perforations 12 are configured to communicate between the inside and outside of the conical section 11. These perforations 12 are used to receive sutures to enable bone portions to be attached to the implant 9. In an alternate embodiment, the operative lower section 10 is provided with perforations communicating between the inside and outside.

The implant 9 of FIG. 15 varies in mechanical construction as compared to the implant of FIG. 3. In an embodiment, the conical section 11 and the operative lower section 10 (of FIGS. 15-19) are formed of a single curved surface extending from top to bottom portion of the implant 9. The implant 9 is made without providing any step 14 between the conical section 11 and the operative lower section 10. Further, the implant 9 also includes a plurality of serrations 15A facing downwards is configured on the operative lower section. The serrations 15A are in the form of vertical ridges provided on the lower section 10 (please refer FIGS. 15-19). These serrations 15A will compress the inner surface of the cancellous bone i.e., the humerus shaft 7 while insertion of the implant 9, and configured to provide a better fixation of the implant 9. Further, the serrations 15A further configured to offer easier removal of the implant 9 whenever needed.

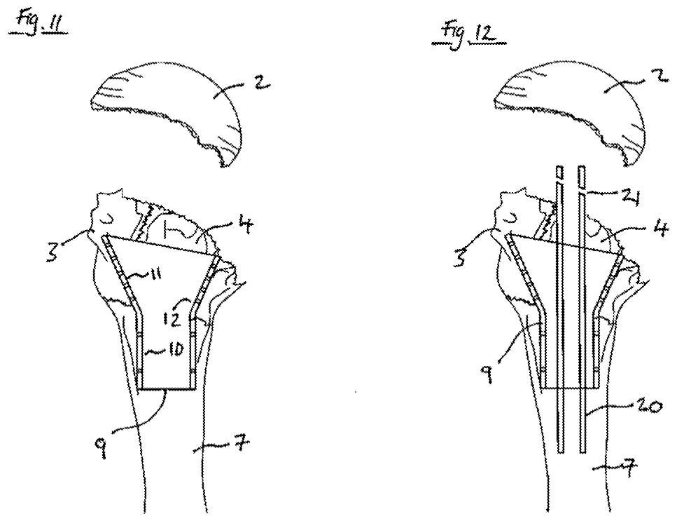

FIG. 8 illustrates, in schematic cross-section, how the implant 9 of FIGS. 3-7C is used in surgery. In FIG. 8A, any loose bone at the proximal end 18 of the fractured humeral shaft 7 may be removed. In FIG. 8B, the internal surface 19 of the humeral shaft 7 has been pared back to match the outside diameter of a humeral repair implant 9. If a circular cross-section variant is being used, this may be accomplished using a reamer. Typically, such a reamer might have a diameter of 8, 9 or 10 mm, with the size chosen to approximately match the outside diameter of the lower section 10 of the implant 9 to be used. In alternative methods, the hole in the shaft 7 of the humerus 1 is made a few mm larger than the outside diameter of the lower section 10, to allow bone cement to be used to secure the implant 9 in place. In this instance, holes may be made through the wall of the lower section 10 of the implant 9 to allow ingress of bone cement to effect a firmer fixation.

For a more trapezoidal cross-section, alternative means of shaping is employed. The implant 9 of FIG. 7C is located in the open end of the shaft 7 of the fractured humerus 1. It can be seen that this embodiment of the implant 9 is also provided with perforations 12 in the conical section 11 of the implant 9. In an embodiment, bone glue may be used to secure the implant 9 in place, and these perforations 12 in the implant 9 assist the glue in securing the implant 9 in place.

FIGS. 9-12 illustrate, schematically, further steps on the repair of the proximal humerus using the implant 9 of the invention. FIG. 9 illustrates the implant 9 in place in the shaft 7 of the humerus 1, ready to receive the other portions of the fractured bone. In FIG. 10, the greater tuberosity 3 has been positioned in its correct place relative to the humeral shaft 7, and resting against the surface of the conical section 11 of the implant 9. The portion of the tuberosity that would otherwise obscure the implant 9 has been omitted, for clarity. When in position, the greater tuberosity 3 can be sutured in place using adjacent perforations 12 in the conical section 11 of the implant 9.

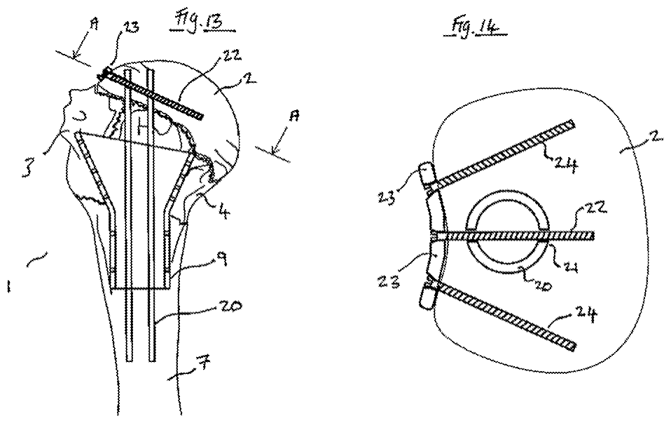

In FIG. 11, the lesser tuberosity 4 has been positioned in its correct place relative to the other anatomical features, again resting on the surface of the conical section 11 of the implant 9. Again, the portion of the tuberosity 3 that would otherwise obscure the implant 9 has been omitted, for clarity. Further, the lesser tuberosity 4 may then be sutured in place using adjacent perforations 12 in the conical section 11 of the implant 9. Now the humeral head 1 is placed on the reconstructed neck. Now a nail 20 is inserted in to the head and threaded through the humeral implant 9 in to the shaft 7. In FIG. 12, a rod 20, (commonly called a “nail” in orthopedic surgery) has been inserted through the lumen of the humeral repair implant 9, and into the interior of the shaft 7 of the humerus 1. The rod 20 is provided with a threaded hole 21 towards its upper end. The threaded hole 21 is preferable angled to approximately the same angle as the anatomical neck 5 of the humerus.

FIGS. 13 and 14 illustrate the final stages of the fracture repair when the patient's own humeral is to be used. Again with the portions of the bone obscuring the implant 9 not illustrated, for clarity. FIG. 14 is a cross-section through the line A—A. The head 2 of the humerus 1 is secured in place on top of the two tuberosities 3, 4. A screw 22 is passed through a plate 23, and through the subchondral bone of the head 2 and into the threaded hole 21 in the rod/nail 20. Further screws 24 may then be similarly inserted through the plate 23, into the head 2, providing further stability. Implants and methods of humeral repair described herein are suitable for use in human surgery or veterinary surgery for animals having a humerus.

However, after reconstruction of the humeral neck with the help of the implant 9, if surgeon feels that humeral head 1 is not salvageable, the current implant 9 offers surgeon the flexibility to replace the humeral head 1 (Hemiarthroplasty). The hollow nature of the implant 9 allows safe passage of the humeral stem, which can be fixed with cement. The conical section 11 of the implant 9 promotes healing of the tuberosities 3, 4 and restores the shoulder function.

Furthermore, should the patient with hemiarthroplasty needs revision surgery, the current implant 9 offers ease of conversion to reverse shoulder replacement.

Thus, the implant 9 of the present disclosure helps in preserving the original anatomical elements of the joint, rather than replacing them.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure described herein above has several technical advantages including, but not limited to, the realization of a humeral repair implant, that:
helps in preserving the original anatomical elements of the joint, rather than replacing them.

The embodiments herein, the various features, and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The disclosure has been described with reference to the accompanying embodiments which do not limit the scope and ambit of the disclosure. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A humeral repair implant for repair of a fracture of the proximal humerus, said implant comprising:
(a) a hollow body open at both ends;
(b) a conical section configured at a top portion of the body; and
(c) an operative lower section extending downwards from said conical section;
wherein a plurality of perforations are defined in said conical section, said perforations being configured to communicate between an inside and outside of said conical section.

2. The humeral repair implant as claimed in claim 1, wherein said operative lower section is tubular.

3. The humeral repair implant as claimed in claim 1, wherein said conical section and said operative lower section are separated by a step.

4. The humeral repair implant as claimed in claim 2, wherein a plane of an open end of said conical section is angled relative to an axis of the operative lower section.

5. The humeral repair implant as claimed in claim 4, wherein said angle is between 15° and 45°.

6. The humeral repair implant as claimed in claim 1, wherein a cross-section of said conical section perpendicular to an axis of said conical section is circular.

7. The humeral repair implant as claimed in claim 1, wherein a cross-section of said conical section perpendicular to an axis of said operative lower section is an isosceles trapezium having rounded vertices.

8. The humeral repair implant as claimed in claim 1, wherein a slot is configured at a bottom open end of the operative lower section that extends towards a step between said conical section and said lower section.

9. The humeral repair implant as claimed in claim 1, wherein an outside surface of operative lower section includes protruding ridges.

10. The humeral repair implant claimed in claim 1, wherein at least a portion of the outwardly-facing surface of said operative lower section is roughened.

11. The humeral repair implant as claimed in claim 1, wherein at least a portion of an outwardly-facing surface of said conical section is a trabecular surface.

12. The humeral repair implant as claimed in claim 1, wherein said conical section and said operative lower section are formed of a single curved surface extending from a top to bottom portion of said implant.

13. The humeral repair implant as claimed in claim 12, wherein said operative lower section includes perforations communicating between the inside and outside.

14. The humeral repair implant as claimed in claim 12, wherein a plurality of serrations facing downwards are configured on said operative lower section.

15. The humeral repair implant as claimed in claim 1, wherein said implant is made from metal, a biocompatible plastics material or a biodegradable material.
